(19) United States
(12) Patent Application Publication
(10) Pub. No.: US 2019/0216440 A1
(43) Pub. Date: Jul. 18, 2019

(54) ULTRASOUND DIAGNOSTIC APPARATUS AND STORAGE MEDIUM

(71) Applicant: Konica Minolta, Inc., Tokyo (JP)

(72) Inventors: Takashi KIMOTO, Yokohama-shi (JP); Jo Shikama, Tokyo (JP)

(21) Appl. No.: 16/243,139

(22) Filed: Jan. 9, 2019

(30) Foreign Application Priority Data
Jan. 18, 2018 (JP) 2018-006064

(51) Int. Cl. A61B 8/00; A61B 8/08

(52) U.S. Cl. CPC A61B 8/54; A61B 8/463; A61B 8/469; A61B 8/465; A61B 8/486

(57) ABSTRACT

An ultrasound diagnostic apparatus includes: a hardware processor which generates sound ray data and displays ultrasound images based on the sound ray data; and an input receiver. The hardware processor stores playback data used for displaying the ultrasound images. The hardware processor performs, in a two-screen display control for displaying a live moving image on the ultrasound images and playback data on the live moving image side by side in a display, changes display control on the live moving image if the input receiver receives a first input operation, and changes display control on the playback image while continuing displaying the live moving image if the input receiver receives a second input operation.

### BACKGROUND

### Technological Field

The present invention relates to an ultrasound diagnostic apparatus and a storage medium.

### Description of the Related Art

Conventionally, there is an ultrasound diagnostic apparatus which generates an ultrasound image reflecting an internal structure of a subject and displays it on a display by irradiating the inside of the subject with an ultrasound from an ultrasound probe, receiving reflected ultrasound (reflection waves), and processing sound ray data generated on the basis of the obtained reception signal. Such an ultrasound diagnostic apparatus is also used as a noninvasive diagnostic apparatus for a medical practice to human beings.

Some ultrasound diagnostic apparatus can display ultrasound images as a real-time live moving image by sequentially updating an ultrasound image to be displayed based on newly generated sound ray data. Further, some ultrasound diagnostic apparatus can display ultrasound images included in the live moving image as a playback image (cine image) on the basis of playback data which is stored in a storage, includes sound ray data on the ultrasound image displayed as the live moving image or processed data obtained by processing the sound ray data (for example, see Japanese Patent Laid-Open Publication No. 11-318903).

In addition, some ultrasound diagnostic apparatus can display two ultrasound images side by side (two-screen display). In such ultrasound diagnostic apparatus, for example, while the live moving image displayed on one of the two screens is stopped, a playback image regarding the live moving image is displayed on the other of the two screens. By changing the frame of the playback image, it is possible to compare the image of the stopped live moving image with the playback image of a desired frame before stopping the live moving image, to store the playback image of the desired frame, and the like. Here, input operations for stopping the live moving image, changing the frame of the playback image, and the like are received by various input receivers such as a touch panel and a console.

### SUMMARY

However, the input receiver of a conventional ultrasound diagnostic apparatus during the two-screen display is used for operating only one of the two screens (active screen), and cannot be used for the other screen (inactive screen). When a live moving image is displayed on one of the two screens, the screen of the live moving image is usually the active screen to be operated. Therefore, for performing operations such as changing the frame of the playback image on the other screen, it is necessary to pause the live moving image and to switch the active screen to be operated to the screen of the playback image. Therefore, there is a problem that ultrasound diagnostic apparatus cannot be used in a flexible way of, for example, operating playback images while displaying a live moving image in an operational state.

An object of the present invention is to provide an ultrasound diagnostic apparatus and a recording medium which enables operation of a playback image while displaying a live moving image in an operable state.

In order to achieve at least one of the above objects, the ultrasound diagnostic apparatus reflecting one aspect of the present invention includes:

a hardware processor which generates sound ray data on ultrasound based on a reception signal from an ultrasound probe, the ultrasound being transmitted from the ultrasound probe to a subject and reflected in the subject and which displays ultrasound images based on the sound ray data on a display; and

an input receiver which receives an input operation for changing display control on the ultrasound images by the hardware processor,

wherein the hardware processor stores the sound ray data used for displaying the ultrasound images or processed data obtained by processing the sound ray data in a storage, as playback data to be used for controlling playback and display of the ultrasound images,

wherein the hardware processor performs two-screen display control for displaying a live moving image and a playback image on the display side by side, the live moving image including ultrasound images being sequentially updated and displayed based on the sound ray data which is newly generated, and the playback image being ultrasound images replayed and displayed based on the playback data on the live moving image, and

wherein, in the two-screen display control,
if the input receiver receives a first input operation for changing display control on the live moving image, the hardware processor changes display control on the live moving image according to the first input operation, and
if the input receiver receives a second input operation for changing display control on the playback image, the hardware processor changes display control on the playback image according to the second input operation while continuing displaying the live moving image.

Further, in order to achieve at least one of the above objects: a non-transitory computer-readable storage medium storing a program causing a computer provided in an ultrasound diagnostic apparatus to perform:

generating sound ray data on ultrasound based on a reception signal from an ultrasound probe, the ultrasound being transmitted from the ultrasound probe to a subject and reflected in the subject, and

controlling to display an ultrasound image based on the sound ray data on a display,

the program causes the computer to perform:

storing the sound ray data used for displaying the ultrasound images or processed data obtained by processing the sound ray data in a storage as playback data which is used for controlling playback and display of the ultrasound images; and

performing two-screen display control for displaying a live moving image and a playback image on the display side by side, the live moving image including ultrasound images being sequentially updated and displayed based on the sound ray data which is newly generated, and the playback image being ultrasound images replayed and displayed based on the playback data on the live moving image, and

wherein, if a predetermined input receiver receives a first input operation for changing display control on the live moving image in the two-screen display control, display control on the live moving image is changed according to the first input operation, and

wherein, if the input receiver receives a second input operation for changing display control on the playback image in the two-screen display control, display control on the playback image is changed according to the second input operation while continuing displaying the live moving image.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a diagram showing a schematic configuration of an ultrasound diagnostic apparatus according to a first embodiment.

FIG. 2 is a block diagram showing a main functional configuration of the ultrasound diagnostic apparatus according to a first embodiment.

FIG. 3 is a diagram showing an example of an ultrasound diagnostic screen displayed on a display.

FIG. 4 is a diagram showing an example of an ultrasound diagnostic screen during two-screen display.

FIG. 5 is a diagram showing an example of an ultrasound diagnostic screen in which display of operation buttons B1 to B3 is deleted.

FIG. 6 is a diagram showing an example of an ultrasound diagnostic screen during two-screen display.

FIG. 7 is a flowchart showing control procedures for ultrasound image display processing.

FIG. 8 is a diagram showing an example of an ultrasound diagnostic screen according to Modification 1.

FIG. 9 is a diagram showing an example of the ultrasound diagnostic screen and an operation input receiver according to Modification 2.

FIG. 10 is a block diagram showing a main functional configuration of the ultrasound diagnostic apparatus according to the second embodiment.

### DETAILED DESCRIPTION OF EMBODIMENTS

Hereinafter, one or more embodiments of the ultrasound diagnostic apparatus and a storage medium according to the present invention will be described with reference to the drawings. However, the scope of the invention is not limited to the disclosed embodiments.

### First Embodiment

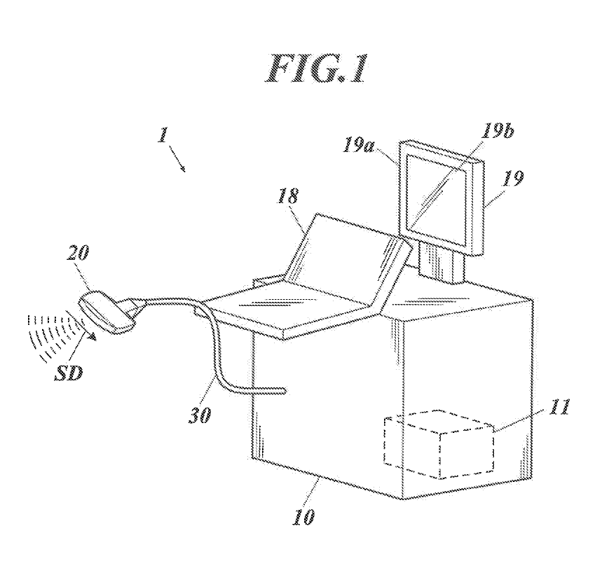

FIG. 1 is a diagram showing a schematic configuration of an ultrasound diagnostic apparatus 1 according to the first embodiment of the present invention.

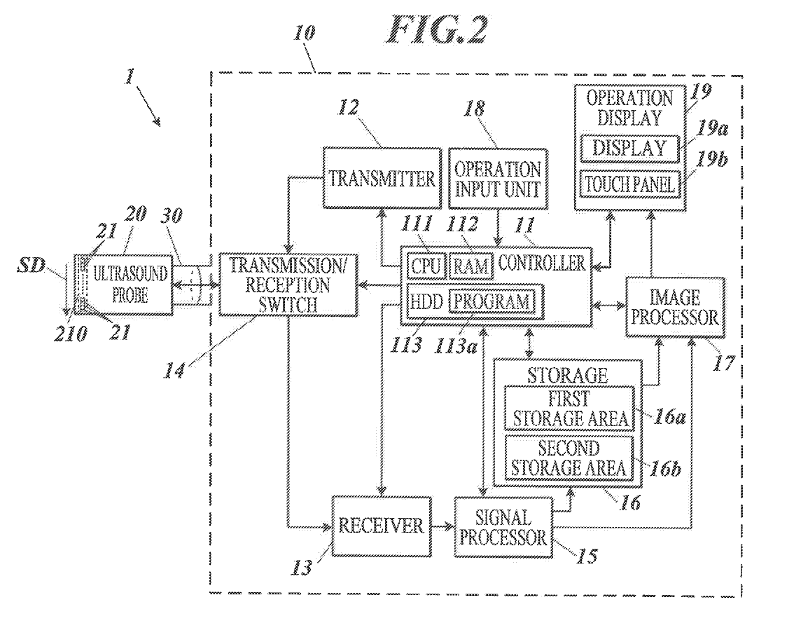

FIG. 2 is a block diagram showing a main functional configuration of the ultrasound diagnostic apparatus 1.

As shown in FIG. 1, the ultrasound diagnostic apparatus 1 includes an ultrasound diagnostic apparatus main body 10 and an ultrasound probe 20 connected to the ultrasound diagnostic apparatus main body 10 via a cable 30. The ultrasound diagnostic apparatus main body 10 is provided with a hardware processor 100 (a computer), an operation input receiver 18 having an operation console, an operation display 19 having a display 19a and a touch panel 19b, and the like. Among them, the operation input unit 18 and the touch panel 19b constitute an “input receiver” which receives an input operation for changing the display control on ultrasound images.

Under the control of the hardware processor 100, the ultrasound diagnostic apparatus 1 outputs a drive signal to the ultrasound probe 20 and causes the ultrasound probe 20 to output ultrasound, on the basis of an operator's input operation on an input device provided on the operation input receiver 18 (for example, a push button, a rotary input key, a toggle switch, a trackball, a keyboard, and a mouse), or an operator's touch operation on the touch panel 19b of the operation display 19. Further, under the control of the hardware processor 100, the ultrasound diagnostic apparatus 1 obtains a reception signal regarding ultrasound reception from the ultrasound probe 20 so as to perform various kinds of processing, and displays ultrasound images based on the reception signal.

As shown in FIG. 2, the ultrasound diagnostic apparatus main body 10 includes a controller 11, a transmitter 12, a receiver 13, a transmission/reception switch 14, a signal processor 15, a storage 16, an image processor 17, the operation input receiver 18, an operation display 19, and the like. The controller 11 and the receiver 13 constitute a sound ray data generating means; the controller 11 and the signal processor 15 constitute a processed data generating means; the controller 11 and the image processor 17 constitute an image data generating means and a display control means; and the controller 11, the signal processor 15, and the image processor 17 constitute a control means. Further, the controller 11, the transmitter 12, the receiver 13, the transmission/reception switch 14, the signal processor 15, the storage 16, and the image processor 17 constitute the hardware processor 100.

The controller 11 includes a CPU 111 (a Central Processing Unit), a RAM 112 (a Random Access Memory), an HDD 113 (a Hard Disk Drive) (non-transitory computer readable recording medium), and the like.

The CPU 111 reads out the program 113a stored in the HDD 113, develops the program 113a in the RAM 112, and totally controls the operation of each part of the ultrasound diagnostic apparatus 1 according to the developed program 113a.

The RAM 112 is a volatile memory such as an SRAM or a DRAM, and provides a working memory space to the CPU 111 to store temporary data.

In addition to the program 113a, the HDD 113 stores various setting data, an image file generated by the ultrasound diagnostic apparatus 1, and the like. Instead of the HDD 113 (or in addition to the HDD 113) may be used a known storage device capable of writing data, such as an SSD (a Solid State Drive).

The transmitter 12 outputs a pulse signal (a drive signal) to be supplied to the ultrasound probe 20 in accordance with a control signal input from the controller 11, so that the ultrasound probe 20 generates ultrasound. The transmitter 12 includes, for example, a clock generation circuit, a pulse generation circuit, a pulse width setting unit, and a delay circuit. The clock generation circuit is a circuit which generates clock signals to determine the transmission timing and the transmission frequency of the pulse signal. The pulse generating circuit is a circuit which generates bipolar rectangular wave pulses having a preset voltage amplitude at a predetermined frequency. The pulse-width setting unit sets the pulse width of the rectangular wave pulses output from the pulse generating circuit. The rectangular wave pulses generated by the pulse generating circuit are separated into different wiring paths for the respective transducers 21 of the ultrasound probe 20 before or after input to the pulse-width setting unit. The delay circuit is a circuit which delays the generated rectangular wave pulses by a delay time set for each wiring route and outputs the delayed pulse depending on the timing of transmission to each of the transducers 21.

The receiver 13 obtains a reception signal input from the ultrasound probe 20 under the control of the controller 11, and generates sound ray data (acoustic line data) based on the reception signal. Here, the sound ray data is data generated based on a reception signal of a series of reflection waves generated by one transmission of the ultrasound. Therefore, the sound ray data includes information on reflection intensity of the ultrasound (strength of the reception signal) at each position in the depth direction of the subject.

The receiver 13 includes, for example, an amplifier, an A/D conversion circuit, and a delay-and-sum circuit. The amplifier is a circuit which amplifies the reception signal corresponding to the ultrasound received by each of the transducers 21 of the ultrasound probe 20 with a predetermined amplification rate. The A/D conversion circuit is a circuit which converts the amplified reception signal into digital data at a predetermined sampling frequency. The delay-and-sum circuit is a circuit which gives a delay time to the A/D converted reception signal for each wiring route corresponding to each of the transducers 21 to adjust the time phase of the signal, and generates the sound ray data by adding (performing phase addition of) the delayed signal.

Under the control of the controller 11, the transmission/reception switch 14 causes the transmitter 12 to transmit a drive signal to the transducers 21 when ultrasound is oscillated from the transducers 21, and performs a switching operation for outputting the reception signal to the receiver 13 when a signal regarding the ultrasound from the transducer 21 is received.

The signal processor 15 performs various kinds of data processing on the ultrasound sound ray data for the purpose of compressing the data amount and adjusting the image quality of the ultrasound image. For example, for displaying a B-mode or M-mode ultrasound image which represents intensity of the reception signal by luminance, the signal processor 15 carries out envelope detection processing, log compression processing, and the like on the sound ray data, so as to adjust gain, dynamic range, and the like, and so as to convert the sound ray data into data representing luminance values. The sound ray data after such processing (hereinafter also referred to as “processed sound ray data”) is output to the storage 16 and the image processor 17. The processed sound ray data is an example of processed data obtained by processing the sound ray data.

The storage 16 is constituted by a volatile memory such as a DRAM (Dynamic Random Access Memory), for example. Alternatively, various nonvolatile memories capable of high-speed rewriting may be used instead of a volatile memory (or in addition to a volatile memory).

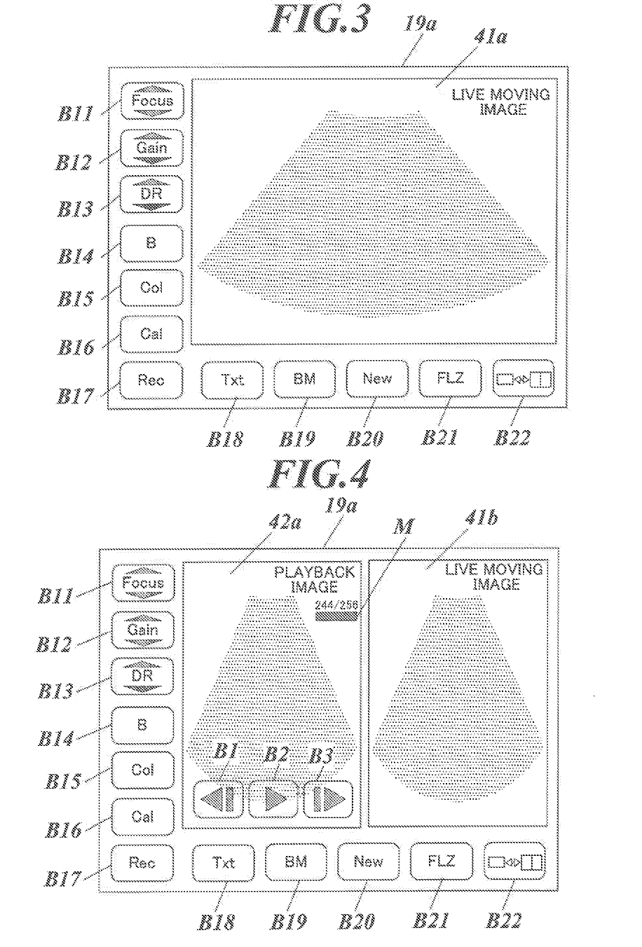

The storage 16 stores the processed sound ray data output from the signal processor 15 for each frame of the ultrasound image. The processed sound ray data stored in the storage 16 is playback data (cine image data) used for controlling playback and display of the ultrasound images of the live moving image described later (such as the first live moving image 41a in FIG. 3) as a playback image (a cine image) (such as a first playback image 42a in FIG. 4). Under the control of the controller 11, the playback data stored in the storage 16 is read as required and output to the image processor 17.

More specifically, the storage 16 has a first storage area 16a and a second storage area 16b. The first storage area 16a and the second storage area 16b each have a storage capacity capable of storing the processed sound ray data of hundreds of frames (256 frames in the present embodiment). The processed sound ray data input to the storage 16 is stored and accumulated in either the first storage area 16a or the second storage area 16b depending on the state of the two-screen display described later. If the amount of the accumulated processed sound ray data exceeds the number of storable frames, the oldest data is overwritten with processed sound ray data which is newly input and is to be stored.

The storage 16 may be configured with hardware common to the RAM 112. That is, the RAM 112 may function as the storage 16. Further, the data stored in the storage 16 as described above may be partly stored in the HDD 113. That is, the HDD 113 may constitute a part of the “storage”.

The image processor 17 performs predetermined image processing on the processed sound ray data output from the signal processor 15 and/or storage 16, thereby generates image data in a format according to the method of display on the display 19a.

The image processor 17 has a DSC (Digital Signal Converter), an image combining unit, and the like.

The DSC performs coordinate transformation, pixel interpolation, frame rate adjustment, and the like on the frame data of the processed sound ray data, so as to convert the above-described frame data according to a coordinate system of the reception signal into frame data (image data) according to a display coordinate system on the display 19a.

The image combining unit generates combined image data by combining the image data of the ultrasound image with data of images (such as an operation button, a body mark, and a scale) to be displayed on the ultrasound diagnostic screen including the ultrasound image.

The functions of the controller 11, the transmitter 12, the receiver 13, the transmission/reception switch 14, the signal processor 15, the storage 16, and the image processor 17 constituting the hardware processor 100 described above may partially or entirely realized by a hardware circuit (an integrated circuit) such as an ASIC (Application Specific Integrated Circuit), an FPGA (Field Programmable Gate Array), an DSP (Digital Signal Processor), or the like. Further, two or more functions of these units may be incorporated in a common integrated circuit.

The operation input receiver 18 is provided with a physical operation unit(s) such as a push button, a rotary input key, a toggle switch, a trackball, a keyboard, a mouse, and the like, converts an input operation on the physical operation unit by an operator into an operation signal, and outputs the operation signal to the controller 11.

The display 19a of the operation display 19 has a display screen and a driving unit thereof. The display screen uses any one of various display methods and may be an LCD (Liquid Crystal Display), an organic EL (Electro-Luminescence) display, an inorganic EL display, a plasma display, a CRT (Cathode Ray Tube) display, or the like. The display 19a generates a drive signal for the display screen (each display pixel) according to the control signal output from the controller 11 and the image data generated by the image processor 17, and displays, on the display screen, menu and status regarding ultrasound diagnosis, an operation button indicating a target of the touch operation received by the touch panel 19b, measurement data on ultrasound images based on the received ultrasound, and the like.

The touch panel 19b of the operation display 19 is an electrostatic capacity type touch panel superimposed on the display screen of the display 19a. On the basis of a change in electrostatic capacitance between the inner conductive film and the surface of the touch panel 19b due to the touch of the operator's fingertip on the surface, the touch panel 19b detects the touch (a touch operation) and outputs a signal indicating the detected position (a coordinate) to the controller 11 as an operation signal. The type of the touch panel 19b is not limited to the electrostatic capacity type but may be another type, such as a resistance film type or an electromagnetic induction type.

These operation input receiver 18 and the operation display 19 may be integrally provided in the housing of the ultrasound diagnostic apparatus main body 10 or may be provided outside of the ultrasound diagnostic apparatus main body 10 via a cable or the like. As long as the ultrasound diagnostic apparatus main body 10 is provided with an operation input terminal and a display output terminal, conventional peripheral devices for operation and display may be connected to these terminals and used.

The operation input receiver 18 and the operation display 19 are separately provided in FIG. 1, however, the operation input receiver 18 and the operation display 19 may be integrally configured. For example, various operation buttons and a trackball of the operation input receiver 18 may be provided on the housing of the operation display 19 which includes the display 19a and the touch panel 19b.

The ultrasound probe 20 oscillates and transmits (emits) ultrasound (here, about 1 to 30 MHz) to a subject such as a living body and also functions as an acoustic sensor for receiving reflection waves (echo), which is the transmitted ultrasound reflected in the subject, and for converting the received reflection waves into an electric signal. The ultrasound probe 20 includes a transducer array 210 which is an array of a plurality of transducers 21 which transmits and receives ultrasound.

The transducer array 210 is an array of a plurality of transducers 21 each having a piezoelectric body including a piezoelectric element which generates an electric charge due to deformation (expansion and contraction) of the piezoelectric body and electrodes provided at both ends of the piezoelectric body. When a voltage pulse (pulse signal) is supplied to a transducer 21, the piezoelectric body is deformed according to the electric field generated in each piezoelectric body and transmits ultrasound. When ultrasound of a predetermined frequency band is incident on the transducers 21, the thickness of the piezoelectric body fluctuates (vibrates) according to the pressure level. As a result, the electric charge corresponding to the fluctuation appears at both ends in the thickness fluctuation direction of the piezoelectric body, and an amount of electric charge corresponding to the electric charge is induced at the electrodes at both ends of the piezoelectric body. A ferroelectric is used as the piezoelectric body here.

The transducer array 210 of the ultrasound probe 20 of the present embodiment includes 192 transducers 21 which are arranged one-dimensionally in the predetermined transducer array direction. Alternatively, the transducers 21 may be arranged two-dimensionally both in the transducer array direction and in the direction orthogonal to the transducer array direction. Further, the number of the transducers 21 may be arbitrarily set. On the basis of the pulse signal from the transmitter 12, the ultrasound probe 20 according to the present embodiment transmits ultrasound from a series of the transducers 21 (for example, 64 transducers 21) among the 192 transducers 21. Every time ultrasound is generated, the series of the transducers 21 transmitting the ultrasound is shifted in the transducer array direction by a predetermined number of transducers 21, so that scanning is performed in the scanning direction SD which is parallel to the transducer array direction. In addition, in the present embodiment, the ultrasound probe 20 of a convex electronic scanning type having a sectoral transmission range of ultrasound transmitted at different timings is used. Alternatively, the ultrasound probe 20 may be of any scanning type including various types of electronic scanning, such as linear electronic scanning and sector electronic scanning, and various types of mechanical scanning, such as linear scanning, sector scanning, arc scanning, and radial scanning. The received frequency of ultrasounds of the ultrasound probe 20 may be set to any bandwidth.

The ultrasound diagnosis apparatus 1 may be an ultrasound diagnostic apparatus main body 10 connected to one of ultrasound probes 20 which are different according to the diagnosis target.

The cable 30 has a connector (not shown) at one end for connection with the ultrasound diagnostic apparatus main body 10. The ultrasound probe 20 is detachably connected to the ultrasound diagnostic apparatus main body 10 via the cable 30.

The various operations for displaying the ultrasound image according to the ultrasound diagnosis apparatus 1 of the present embodiment will now be described.

When a predetermined operation for instructing start of inspection is input to the ultrasound diagnostic apparatus 1, the ultrasound probe 20 starts a transmission/reception operation of ultrasound with the setting according to the instructed inspection. Sound ray data representing internal structures of the subject, processed sound ray data, and image data on the ultrasound image are sequentially generated on the basis of the reception signal received by the ultrasound probe 20, and an ultrasound diagnostic screen including the ultrasound image is displayed on the display 19a.

FIG. 3 is a diagram showing an example of the ultrasound diagnostic screen displayed on the display 19a.

In the ultrasound diagnostic screen in FIG. 3 is displayed a real-time first live moving image 41a (hereinafter, in the case where the order is not considered, it is simply written as a live moving image 41) including ultrasound images sequentially updated and displayed based on newly generated sound ray data.

The ultrasound image of the live moving image 41 is displayed on the display 19a through generation of sound ray data at the receiver 13 based on the newly received reception signal, processing of the sound ray data in the signal processor 15, conversion of the processed sound ray data into image data in the image processor 17, and input of the image data to the display 19a. By continuously performing these operations and sequentially updating the ultrasound image, the live moving image 41 is displayed. The frame number of the ultrasound images displayed per second in the live moving image 41 is approximately several tens to hundreds frames/second. The narrower the displayed area of the ultrasound image (that is, the scanning area and the reach depth of the ultrasound) is, the more the number of the frame is.

The processed sound ray data used for display of the first live moving image 41a is stored and accumulated in the first storage area 16a of the storage 16, as first playback data.

Further, in the ultrasound diagnostic screen of FIG. 3, as well as the first live moving image 41a, multiple operation buttons B11 to B22 (an operation target image(s)) are displayed. The operation target image(s) each indicates a target on which the touch operation (an input operation) received by the touch panel 19b is performed. Among the buttons, the operation buttons B11 to B17 and the operation buttons B18 to B22 are respectively arranged on the left side of the first live moving image 41a and under the first live moving image 41a.

The operation button B11 showing the character “Focus” is a button for adjusting the focus depth of the ultrasound to be transmitted.

The operation button B12 showing the character “Gain” is a button for adjusting the level of the reception signal so that the range of the reception signal used for displaying the ultrasound image is appropriate.

The operation button B13 showing the character “DR” is a button for adjusting the extent of range (dynamic range) of the reception signal level used for displaying the ultrasound image.

The operation button B14 showing the character “B” is a button for displaying the ultrasound image with B-mode for representing intensity of the reception signal by luminance.

The operation button B15 showing the character “Col” is a button for displaying an ultrasound image by Color Doppler Imaging, in which the movement speed and the movement direction are represented by colors in the target area.

The operation button B16 showing the character “Cal” is a button for performing transition to a measurement mode for measuring the distance between two points in the ultrasound image.

The operation button B17 showing the character “Rec” is a button for generating and storing moving image data by recording a predetermined area in the ultrasound diagnostic screen.

The operation button B18 showing the character “Txt” is a button for performing transition to a text input mode for inputting text on the ultrasound image.

The operation button B19 showing the character “BM” is a button for displaying a body mark in the ultrasound image. Here, the body mark is, for example, an image including a figure indicating the part of the subject and a probe mark disposed at a position corresponding to the inspection portion in the figure and indicating the arrangement direction of the ultrasound probe 20 (probe direction) at the inspection portion.

The operation button B20 showing the character “New” is a button for stopping the current ultrasound scanning to start a new inspection.

The operation button B21 showing the character “FLZ” is a button for stopping the first live moving image 41a at the operation timing of the operation button B21 to display a still image (a freeze image).

The operation button B22 is a button for performing transition to two-screen display to display two ultrasound images side by side.

According to the ultrasound diagnostic apparatus 1 of the present embodiment, when a touch operation for selecting the operation button B22 is performed on the ultrasound diagnosis screen in FIG. 3, the transition to the two-screen display is performed so that two ultrasound images are displayed side by side. On one of the two screens is displayed a second live moving image 41b corresponding to the current ultrasound scanning. On the other screen is displayed the first playback image 42a (a still image) which is a replayed and displayed ultrasound image included in the first live moving image 41a (hereinafter, in the case where the order is not considered, it is simply written as a playback image 42). Among the ultrasound images included in the first live moving image 41a, the one at the time when the operation button B22 is touched is displayed at the start of the display of the first playback image 42a.

For the sake of convenience, the first live moving image 41a and the second live moving image 41b are distinguished from each other in order to identify a portion (the first live moving image 41a) corresponding to the first playback image 42a in the sequential live moving image 41. They do not have to be separate live moving images having different diagnostic parts or the like. In other words, the first live moving image 41a and the second live moving image 41b may be different portions in one sequential live moving image 41 generated based on a series of ultrasound scanning operation.

In the ultrasound diagnostic screen in FIG. 3, the transition to the two-screen display may also be performed when the operation button B21 for stopping the first live moving image 41a is selected.

FIG. 4 is a diagram showing an example of an ultrasound diagnostic screen during two-screen display.

In the ultrasound diagnostic screen shown in FIG. 4, the first playback image 42a regarding the first live moving image 41a is displayed on the left half of the screen, and the new second live moving image 41b is displayed on the right half of the screen. Further, on the first playback image 42a in the ultrasound diagnostic screen are superimposed operation buttons B1 to B3, which receive input operations (second input operation) for changing display control on the first playback image 42a. As shown in FIG. 4, the operation buttons B1 to B3 may be translucent such that the first playback image 42a behind the operation buttons B1 to B3 can also be visually recognized

The frame of the ultrasound image displayed as the first playback image 42a can be changed. That is, besides the ultrasound image of a frame at the stop timing due to the touch operation on the operation button B22, it is possible to select and display one of the ultrasound images regarding the first playback data of 256 frames stored in the first storage area 16a of the storage 16 (i.e., 256 frames immediately before the first live moving image 41a is stopped). Specifically, an ultrasound image of the previous frame can be displayed each time the touch operation is performed on the operation button B1, and an ultrasound image of the next frame can be displayed each time the touch operation is performed on the operation button B3.

Instead of the touch operations on the operation buttons B1 to B3, the frame of the first playback image 42a may be changed in accordance with a tap operation (an operation of quickly touching the touch panel 19b with a finger etc. and then releasing the finger etc. from the touch panel 19b) on an area where the touch panel 19b is superimposed on the first playback image 42a, a flick operation (an operation of quickly sliding a finger etc. on the touch panel 19b while touching the touch panel 19b with finger etc.), and the direction of the flick operation. For example, an ultrasound image of the next frame may be displayed by a flick operation in the right direction, and an ultrasound image of the previous frame may be displayed by a flick operation in the left direction. An ultrasound image of the next frame may be displayed by the tap operation on the right half area in the first playback image 42a, and the ultrasound image of the previous frame may be displayed by the tap operation on the left half area in the first playback image 42a. If the tap operation or the flick operation can be performed in this way, the display of the operation buttons B1 to B3 may be omitted.

Further, when a touch operation is performed with respect to the operation button B2, the display of the moving image of the first playback image 42a (motion playback image) may be started by sequentially displaying and updating ultrasound images of multiple frames. When the display of all the frames to be displayed as the moving image of the first playback image 42a is completed, the first frame is displayed again and the moving image of the first playback image 42a is repeatedly displayed. Further, when a touch operation is performed on the operation button B1 or B3 during display of the moving image of the first playback image 42a, the playback speed (the number of displayed frames per second) of the moving image of the first playback image 42a may be reduced or increased. The upper limit of the playback speed of the moving image of the first playback image 42a is set to the same number of frames per second as that of the first live moving image 41a, and the lower limit is set to a predetermined value according to the setting in the ultrasound diagnostic apparatus 1. Further, by performing a touch operation on the operation button B2 during display of the moving image of the first playback image 42a, the display of the moving image of the first playback image 42a is stopped and a still image of the first playback image 42a can be displayed.

The playback of the moving image of the first playback image 42a may be started or stopped, or the playback speed may be changed in accordance with a tap operation on an area where the touch panel 19b is superimposed on the first playback image 42a, a flick operation, and the direction of the flick operation, instead of the touch operations on the operation buttons B1 to B3. For example, the display of the moving image of the first playback image 42a may be started by a flick operation in the right direction, and the display of the moving image of the first playback image 42a may be stopped by a flick operation in the left direction. Alternatively, during the display of the moving image of the first playback image 42a, a flick operation may be performed to change the playback speed of the moving image. For example, the playback speed may be increased by a flick operation in the right direction, and the playback speed may be decreased by a flick operation in the left direction. The playback speed may be increased by the tap operation on the right half area in the first playback image 42a, and the playback speed may be decreased by the tap operation on the left half area in the first playback image 42a. If the tap operation or the flick operation can be performed in this way, the display of the operation buttons B1 to B3 may be omitted during the display of the moving image of the first playback image 42a.

If a touch operation is performed on the operation button B17 while the moving image of the first playback image 42a is displayed, image data is generated on moving images in a predetermined area in the ultrasound diagnostic screen including the moving images of the second live moving image 41b and the first playback image 42a. The generated image data is stored in the storage 16 or the HDD 113. For example, in the ultrasound diagnostic screen in FIG. 4, the predetermined area described above may be a partial area including the display areas of the live moving image 41b and the playback image 42a, but not including the display areas of the operation buttons B11 to B22. However, the present invention is not limited to this. The predetermined area described above may be the entire ultrasound diagnostic screen.

In order to generate image data of this moving image, a predetermined compression processing (for example, a compression processing of the Motion JPEG system) may be performed on a series of image data generated by the image processor 17 and output to the display 19a.

In addition, on the first playback image 42a is superimposed a playback position mark M indicating the currently displayed frame among the frames to be replayed. Further, in the present embodiment, by performing a touch operation for specifying two points in the playback position mark M, it is possible to define the playback range of the moving image of the first playback image 42a. When the touch operation is performed on the operation button B2 while the playback range is defined, the moving image of the first playback image 42a in the playback range is repeatedly displayed.

Further, if no touch operation on the touch panel 19b is performed for a predetermined time, the display of the operation buttons B1 to B3 is deleted (disappears) as shown in FIG. 5. As a result, the visibility of the first playback image 42a is improved.

If the touch operation is performed on the first playback image 42a or on an area superimposed on the vicinity of the first playback image 42a on the touch panel 19b while the operation buttons B1 to B3 are not displayed, the operation buttons B1 to B3 are displayed again, as shown in FIG. 4.

Since the input receiver (operation buttons B1 to B3) to receive the second input operation(s) is provided for operating the playback image 42 in the ultrasound diagnostic apparatus 1 of the present embodiment in this way, various operations on the first playback image 42a can be performed while display of the second live moving image 41b is continued. Further, since input operation(s) (first input operation(s)) on the operation buttons B11 to B22 is valid even during the two-screen display, various operations for changing display control on the second live moving image 41b can also be performed by operation on the operation buttons B11 to B22.

The second playback data (the processed sound ray data) on the second live moving image 41b displayed on the ultrasound diagnostic screen in FIG. 4 is stored and accumulated in the second storage area 16b of the storage 16. That is, the processing to read out the first playback data in the first storage area 16a and to display the first reprocessed image 42a is performed in parallel with the processing to store the second playback data on the second live moving image 41b in the second storage area 16b.

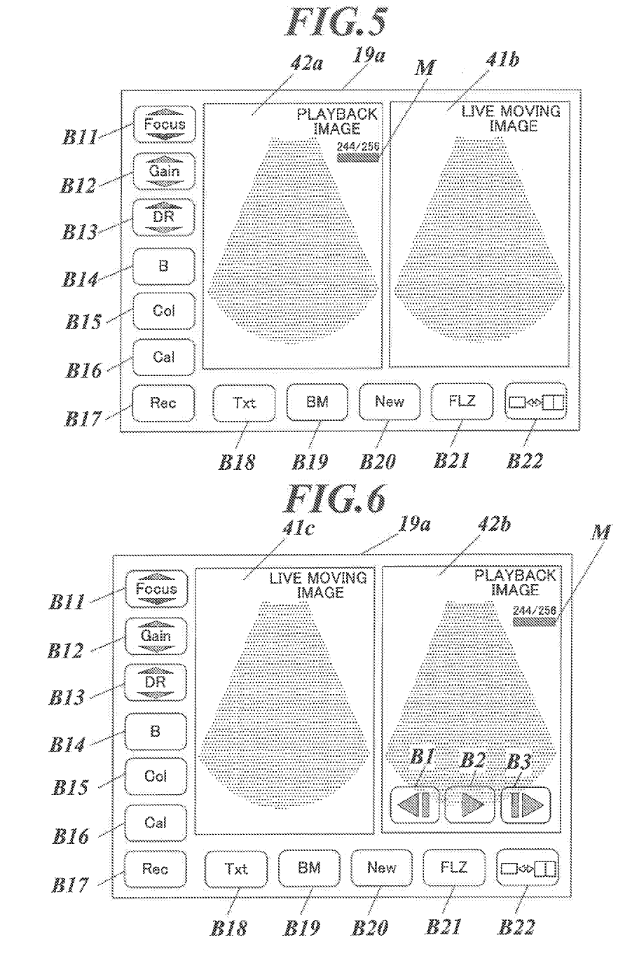

If a touch operation is performed on the operation button B21 showing the character “FLZ” in the ultrasound diagnosis screen in FIG. 4, the second live moving image 41b on the right screen is stopped at the timing of the touch operation and is switched to the second playback image 42b as shown in FIG. 6. The operation buttons B1 to B3 and the playback position mark M are superimposed and displayed on the second playback image 42b, and operations such as frame change and motion playback can be performed on the second playback image 42b, as well as on the above-described first playback image 42a. The processing to display the second playback image 42b is performed on the basis of the second playback data stored in the second storage area 16b of the storage 16.

Meanwhile, on the left screen, the display of the first playback image 42a is stopped and is switched to display of a new third live moving image 41c. In the third live moving image 41c, the display of the operation buttons B1 to B3 and the playback position mark M is deleted.

If the display of the third live moving image 41c is started, the first playback data on the first live moving image 41a stored in the first storage area 16a of the storage 16 is deleted. Then, third playback data (the processed sound ray data) on the third live moving image 41c is stored and accumulated in the first storage area 16a. In this way, each time the touch operation on the operation button B21 is performed to stop the current live moving image 41 (to start display of playback image) and to start display of a new live moving image 41, the target area for storing the playback data is switched between the first storage area 16a and the second storage area 16b. As a result, the playback data on the latest live moving image 41 is always accumulated, and the display of the playback image 42 regarding the latest live moving image 41 can be started at any time.

Next, control procedures for ultrasound image display processing of the controller 11 regarding display of ultrasound images will be described. The operation subjects performing the ultrasound image display processing are the controller 11, the receiver 13, the signal processor 15, and the image processor 17, however, the hardware processor 100 will be described as an operation subject for convenience.

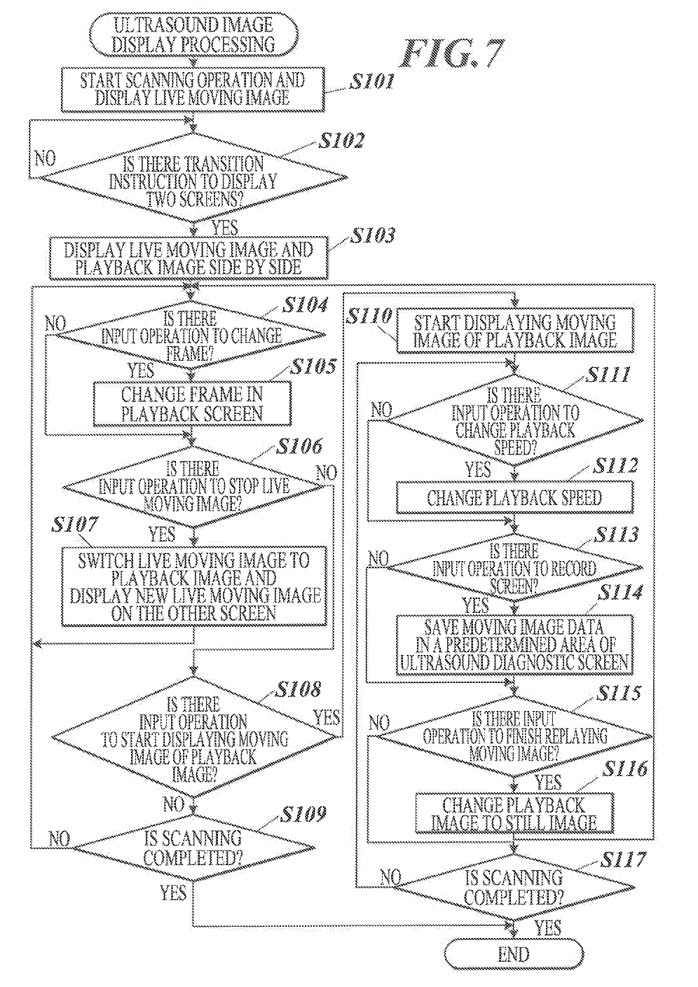

FIG. 7 is a flowchart showing control procedures for the ultrasound image display processing.

After designating an inspection target part, setting scanning operation of ultrasounds according to the inspection part, and setting parameters (gain, dynamic range, etc.) used for processing the reception signal, for example, the ultrasound image display processing is started if a predetermined input operation for starting ultrasound scanning is performed.

When the ultrasound image display processing is started, the hardware processor 100 starts the scanning operation and displays the first live moving image 41a on the basis of the obtained reception signal (step S101, FIG. 3). Further, the hardware processor 100 starts control to store the first playback data on the first live moving image 41a in the first storage area 16a of the storage 16.

The hardware processor 100 determines whether or not an instruction on transition to the two-screen display has been made, that is, whether or not a touch operation on the operation button B22 has been performed (step S102). When it is determined that the touch operation has been performed (“YES” in step S102), the two-screen display control is performed so that the first playback image 42a regarding the first live moving image 41a and the new second live moving image 41b are displayed side by side (step S103, FIG. 4). Further, the hardware processor 100 starts control to store the second playback data on the second live moving image 41b in the second storage area 16b of the storage 16.

If it is determined that on instruction on transition to two-screen display has been made (“NO” in step S102), the hardware processor 100 executes the processing of step S102 again. Although not shown in FIG. 7, if an input operation for instructing the completion of ultrasound scanning (a touch operation of selecting the operation button B20) is performed before the instruction on transition to the two-screen display, the hardware processor 100 completes the ultrasound image display processing.

The hardware processor 100 determines whether or not an input operation for changing the frame of the first playback image 42a (a touch operation for selecting the operation button B1 or B3, a tap operation, a flick operation, etc.) has been performed (step S104). If it is determined that the input operation has been performed (“YES” in step S104), the frame of the first playback image 42a is changed according to the input operation (step S105). If it is determined that the input operation has not been performed (“NO” in step S104), the hardware processor 100 proceeds to the processing of step S106.

The hardware processor 100 determines whether or not an input operation for stopping the second live moving image 41b (a touch operation of selecting the operation button B21) has been performed (step S106). If it is determined that the input operation has been performed (“YES” in step S106), the hardware processor 100 changes the second live moving image 41b to the second playback image 42b, and starts display of a new third live moving image 41c in the other screen (Step S107, FIG. 6). The hardware processor 100 further deletes the first playback data stored in the first storage area 16a of the storage 16 and starts control to store the third playback data on the third live moving image 41c in the first storage area 16a. After the completion of step S107, the hardware processor 100 proceeds to the processing of step S104.

If it is determined that the input operation for stopping the second live moving image 41b has not been performed (“NO” in step S106), the hardware processor 100 determines whether or not an input operation for starting display of the moving image of the first playback image 42a (an input operation of selecting the operation button B2, a flick operation, and the like) has been performed (step S108). If it is determined that the input operation has not been performed (“NO” in step S108), the hardware processor 100 determines whether or not the input operation for instructing the completion of ultrasound scanning (a touch operation of selecting the operation button B20) has been performed (step S109). If the completion of scanning has not been instructed (“NO” in step S109), the hardware processor 100 proceeds to the processing of step S104. If the completion of scanning has been instructed (“YES” in step S109), the hardware processor 100 completes the ultrasound image display processing.

If it is determined that the input operation for starting display of the moving image of the first playback image 42a has been performed (“YES” in step S108), the hardware processor 100 starts display of the moving image of the first playback image 42a (step S110).

The hardware processor 100 determines whether or not an input operation for changing the playback speed of the moving image of the first playback image 42a (a touch operation on the button B1 or B3 or a flick operation) has been performed (step S111). If it is determined that the input operation has been performed (“YES” in step S111), the hardware processor 100 changes the playback speed according to the content of the input operation (step S112). If it is determined that the input operation has not been performed (“NO” in step S111), the hardware processor 100 proceeds to the processing of step S113.

The hardware processor 100 determines whether or not an input operation for recording a predetermined area on the ultrasound diagnostic screen as a moving image (a touch operation on the operation button B17) has been performed (step S113). If it is determined that the input operation has been performed (“YES” in step S113), the hardware processor 100 generates image data on a moving image of the predetermined area on the display screen including the second live moving image 41b and the first playback image 42a and stores the image data in the storage 16 or the HDD 113 (step S114). If it is determined that the input operation has not been performed (“NO” in step S113), the hardware processor 100 proceeds to the processing of step S115.

The hardware processor 100 determines whether or not an input operation for completing the display (motion playback) of the moving image of the first playback image 42a (an input operation of selecting the operation button B2 or a flick operation) has been performed (step S115). If it is determined that the input operation has been performed (“YES” in step S115), the hardware processor 100 switches the moving image of the first playback image 42 a to a still image (step S116) and proceeds to the processing of step S104.

If it is determined that the input operation for completing the motion playback has not been performed (“NO” in step S115), the hardware processor 100 determines whether or not an input operation for instructing the completion of ultrasound scanning (a touch operation of selecting the button B20) has been performed (step S117). If completion of scanning has not been instructed (“NO” in step S117), the hardware processor 100 proceeds to the processing of step S111. If completion of scanning has been instructed (“YES” in step S117), the hardware processor 100 completes the ultrasound image display processing.

Although not shown in the flowchart of FIG. 7, if no touch operation on the touch panel 19b has been performed for a predetermined time in the processing of each of the steps S103 to S117, the hardware processor 100 performs control to delete the display of the operation buttons B1 to B3 (FIG. 5). If a touch operation on the touch panel 19b is performed after the deletion, the hardware processor 100 performs control to display the operation buttons B1 to B3 again (FIG. 4).

### (Modification 1)

Next, Modification 1 of the above embodiment will be described. Modification 1 is different from the above embodiment in the display positions of the operation buttons B1 to B3 which receive the second input operation for changing display control on the playback image 42.

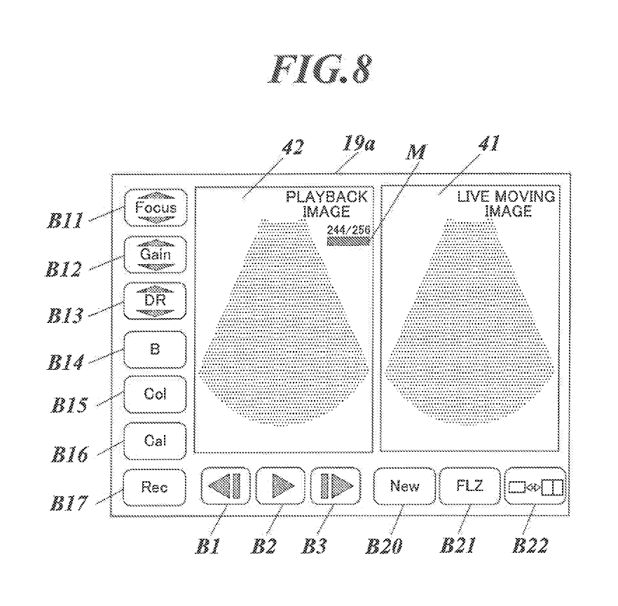

FIG. 8 is a diagram showing an example of ultrasound diagnostic screen according to Modification 1.

As shown in FIG. 8, according to Modification 1, the operation buttons B1 to B3 are superimposed and displayed not on the playback image 42 but on the vicinity of the playback image 42 (so that no image is present between the playback image 42 and the operation buttons B1 to B3). When the operation buttons B1 to B3 are arranged in this way, it is also possible to intuitively and easily perform the input operation for changing the display control on the playback image 42.

According to Modification 1, it is also possible to simultaneously perform various operations for changing display control on the live moving image 41 by the input operation (first input operation) on the buttons B11 to B17 and B20 to B22.

### (Modification 2)

Next, Modification 2 of the above embodiment will be described. Modification 2 is different from the above embodiment in that operations of the live moving image 41 and playback image 42 are performed via the operation console of the operation input receiver 18.

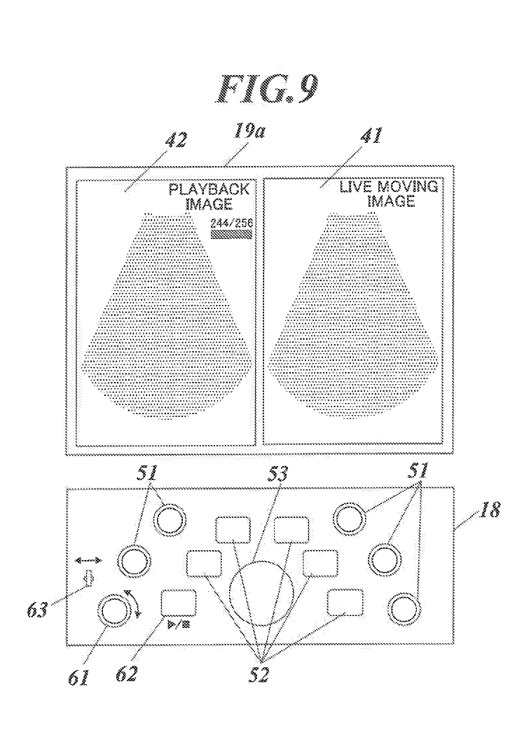

FIG. 9 is a diagram showing an example of the ultrasound diagnostic screen and the operation input receiver 18 according to Modification 2.

In Modification 2, operation buttons are not displayed on the ultrasound diagnostic screen.

Instead, functions as the input receiver for changing the display control on live moving image 41 and playback image 42 are assigned to various physical operation units on the operation console of the operation input receiver 18.

More specifically, the functions as the input receiver which receives input operations for changing the display control on the live moving image 41 are assigned to a rotary input key 51 capable of receiving an input operation of rotation, a push button 52 capable of receiving an input operation of pressing down, and a track ball 53.

Further, in the vicinity of the lower left corner of the operation input receiver 18 are provided a rotary input key 61, a push button 62, and a toggle switch 63 which performs an input operation by tilting to one side and the other side along a predetermined direction (here, along the left-right direction). The functions as the input receiver which receives the input operations for changing the display control on the playback image 42 are assigned to them.

More specifically, by performing an input operation of rotating the rotary input key 61, it is possible to change the frame of the playback image 42 to be displayed according to the rotation amount and the rotation direction of the rotary input key 61. By performing an input operation of tilting the toggle switch 63, it is possible to change the frame of the playback image 42 to be displayed according to the tilt of the toggle switch 63 and the direction of the tilt. By performing an input operation of pressing the push button 62, it is possible to start or stop the display of the moving image of the playback image 42.

The functions assigned to the rotary input key 61, the push button 62, and the toggle switch 63 are not limited to those described above. For example, while the moving image of the playback image 42 is displayed, the playback speed of the moving image may be changed by an input operation of rotating the rotary input key 61.

Further, the configuration of the physical operation unit used for changing the display control on the playback image 42 is not limited to the above. For example, start or stop of the playback of the moving image may be assigned to a push button that can be pushed down may be provided at the upper portion (tip) of the rotary input key 61.

Further, if the playback image 42 is not displayed on the display 19a, the physical operation unit used for changing the display control on the playback image 42 may receive an input operation regarding a predetermined function other than the change of display control on the playback image 42. For example, if a playback image 42 is not displayed, the rotary input key 61 may be assigned for adjustment of gain, focus, dynamic range, etc. of the ultrasound image, and the push button 62 may be used as an execution key for automatic adjustment of gain etc.

As described above, the ultrasound diagnostic apparatus 1 according to the present embodiment includes a hardware processor 100 which generates sound ray data based on a reception signal from an ultrasound probe 20, the sound ray data being on ultrasound transmitted from the ultrasound probe 20 to a subject and reflected in the subject and which displays ultrasound images based on the sound ray data on a display 19a; and an operation input receiver 18 and a touch panel 19b as an input receiving means which receives an input operation for changing display control on the ultrasound images by the hardware processor 100. The hardware processor 100 stores the sound ray data used for displaying the ultrasound images or processed sound ray data obtained by processing the sound ray data in a storage 16, as playback data to be used for controlling playback and display of the ultrasound images. The hardware processor 100 performs two-screen display control for displaying a live moving image 41 and a playback image 42 on the display 19a side by side, the live moving image 41 including ultrasound images being sequentially updated and displayed based on the sound ray data which is newly generated, and the playback image being ultrasound images replayed and displayed based on the playback data on the live moving image 41. In the two-screen display control, if the input receiver receives a first input operation for changing display control on the live moving image 41, the hardware processor 100 changes display control on the live moving image 41 according to the first input operation, and if the input receiver receives a second input operation for changing display control on the playback image 42, the hardware processor 100 changes display control on the playback image 42 according to the second input operation while continuing displaying the live moving image 41.

According to such a configuration, in the two-screen display where the live moving image 41 and the playback image 42 are displayed side by side, it is possible to operate the playback image by the second input operation during display of the live moving image 41 which is in an operable state by the first input operation. As a result, for example, when a live moving image 41 of an affected part is compared with a playback image 42 of a healthy part for diagnosis, the ultrasound diagnostic apparatus 1 can be used in a flexible way of, for example, changing the frame of the playback image 42 of the healthy part or displaying the moving image of the playback image 42 according to the state of the live moving image 41, while adjusting the diagnostic position and image quality of the live moving image 41 of the affected part.

Further, the hardware processor 100 displays one of the ultrasound images of multiple frames based on the playback data as the playback image 42, and if the input receiver receives the second input operation for changing frame of the playback image 42, the hardware processor 100 changes frame of the playback image 42 to be displayed according to the second input operation. As a result, while changing the frame of the playback image 42, a diagnosis can be made by comparing the playback image 42 and the live moving image 41.

Further, the hardware processor 100 displays a moving image of the playback image 42 by sequentially updating an image to be displayed among the ultrasound images of multiple frames based on the playback data. As a result, in the diagnosis by comparing the live moving image 41 and the playback image 42, it is possible to display a moving image of the playback image 42 if necessary.

Further, if the input receiver receives the second input operation for changing playback speed of a moving image of the playback image 42, the hardware processor 100 changes playback speed of a moving image of the playback image 42 according to the second input operation. As a result, in the diagnosis comparing the live moving image 41 and the playback image 42, it is possible to easily adjust the playback speed of the moving image of the playback image 42.

Further, if the input receiver receives the second input operation for specifying a playback range of a moving image of the playback image 42, the hardware processor 100 displays an moving image of the playback image 42 within the playback range according to the second input operation. As a result, a desired playback range of the moving image of the playback image 42 can be replayed and compared with the live moving image 41.

Further, if the input receiver receives a predetermined input operation under the two-screen display control, the hardware processor 100 stores, in the storage 16 or the HDD 113, image data of moving images including a moving image of the live moving image 41 and a moving image of the playback image 42. As a result, it is possible to save the diagnostic screen in which the live moving image 41 and the moving image of the playback image 42 are compared and to refer to it at a desired timing.

Further, the input receiver has a touch panel 19b superimposed on a display screen of the display 19a, and the second input operation is a touch operation on the touch panel 19b. As a result, it is possible to change the display control on the playback image 42 by an intuitive and easy input operation and without looking away from the display screen.

Further, the hardware processor 100 changes a frame of the playback image 42 to be displayed according to a flick operation and a direction of the flick operation. The flick operation is performed on an area where the touch panel 19b is superimposed on the playback image 42. As a result, it is possible to change a frame of the playback image 42 by an intuitive and easy input operation.

Further, the hardware processor 100 starts or stops display of a moving image of the playback image 42 according to a flick operation and a direction of the flick operation. The flick operation is performed on an area where the touch panel 19b is superimposed on the playback image 42. As a result, it is possible to start or stop display of the playback image 42 by an intuitive and easy input operation.

Further, the hardware processor 100 changes playback speed of a moving image of the playback image 42 according to a flick operation and a direction of the flick operation. The flick operation is performed on an area where the touch panel 19b is superimposed on the playback image 42. As a result, it is possible to change playback speed of moving image of the playback image 42 by an intuitive and easy input operation.

Further, the hardware processor 100 displays operation buttons B1 to B3 and B11 to B22 on the display 19a which each indicate a target on which the contact operation is performed, and the second input operation is a touch operation on the operation buttons B1 to B3. According to the embodiment in which the operation buttons are displayed on the display 19a in this way, it is possible to display the operation buttons B1 to B3 indicating easily understood display contents in easily operable positions by the operator. As a result, it is possible to operate the playback image 42 by a more intuitive and easier input operation.

Further, the hardware processor 100 displays the operation buttons B1 to B3 in a vicinity of the playback image 42. As a result, it is possible to grasp the positions of the operation buttons B1 to B3 easily and to operate the playback image 42 without looking away from the display screen.

Further, the hardware processor 100 displays the operation buttons B1 to B3 in a state superimposed on the playback image 42. As a result, it is possible to grasp the positions of the operation buttons B1 to B3 more easily and to operate the playback image 42 without looking away from the display screen.

Further, if a touch operation on the touch panel has not been performed for a predetermined time, the hardware processor 100 deletes the display of the operation buttons B1 to B3. As a result, it is possible to improve the visibility of the playback image 42.

Further, if a touch operation is performed on an area superimposed on the playback image 42 or on a vicinity of the playback image 42 in the touch panel 19b while the operation buttons B1 to B3 are not displayed, the hardware processor 100 displays the operation buttons B1 to B3. As a result, it is possible to improve the visibility of the playback image 42 and to operate the playback image 42 intuitively and easily.

Further, if display of the playback image 42 is deleted or if a display of the playback image 42 is switched to a display of another image, the hardware processor 100 deletes the operation buttons B1 to B3. As a result, it is possible to reduce occurrence of a malfunction that the correspondence relation is hard to understand between the operation buttons B1 to B3 and the playback image 42 which can be operated by the operation buttons B1 to B3.

Further, according to Modification 2, the input receiver of the ultrasound diagnostic apparatus 1 has a rotary input key 61 capable of receiving the input operation of rotation, and, if the second input operation of rotating the rotary input key 61 is received, the control means changes a frame of the playback image 42 to be displayed according to a rotation amount and a rotation direction of the rotary input key 61. As a result, it is possible to change a frame of the playback image 42 by an intuitive and easy input operation.

Further, according to Modification 2, the input receiver of the ultrasound diagnostic apparatus 1 has a toggle switch 63 capable of receiving the input operation of tilting to one side and the other side along a predetermined direction, and if the second input operation of tilting the toggle switch 63 is received, the control means changes a frame of the playback image 42 to be displayed according to a tilt of the toggle switch 63 and a direction of the tilt. As a result, it is possible to change a frame of the playback image 42 by an intuitive and easy input operation.

Further, according to Modification 2, the input receiver of the ultrasound diagnostic apparatus 1 has a push button 62 capable of receiving the input operation of pressing down, and if the second input operation of pressing the push button 62 is received, the control means starts or stops display of a moving image of the playback image 42 according to pressing of the push button 62. As a result, it is possible to start or stop display of a moving image of the playback image 42 by an intuitive and easy input operation.

Further, according to Modification 2, the input receiver of the ultrasound diagnostic apparatus 1 has a physical operation unit including at least one of a rotary input key 61, a toggle switch 63, and a push button 62, and if the playback image 42 is not displayed on the display 19a, the physical operation unit receives an input operation regarding a predetermined function other than the second input operation. As a result, it is possible to reduce the number of the physical operation units in the operation input receiver 18 to a necessary minimum number, to realize an operation input receiver 18 which is simple and easy to understand, and to realize downsizing and cost reduction of the operation input receiver 18.

Further, the hardware processor 100 generates the processed data by performing data processing on the sound ray data, the data processing regarding at least one of compression of data amount and adjustment of image quality of the ultrasound images, generates image data on the ultrasound images on a basis of the processing data, displays the ultrasound images on the display 19a on a basis of the image data, and stores the processed data in the storage 16 as the playback data. By storing the processed sound ray data after the processing by the signal processor 15 as the processing data generating means as playback data in this way, it is possible to reduce the amount of the playback data and to generate playback data allowing display of the playback image 42 having the same image quality as the live moving image 41. Further, since the processed data before conversion into the image data by the image processor 17 is stored as playback data, it is possible to store playback data to display the playback image 42 with little degradation of image quality.

Further, the hardware processor 100 stores a first playback data on a first live moving image 41a in a first storage area 16a in the storage 16, stores a second playback data on a second live moving image 41b in a second storage area 16b which is different from the first storage area 16a in the storage 16, the second live moving image 41b being displayed while the first playback image 42a is displayed on a basis of the first playback data, and if the input receiver receives a predetermined input operation for stopping display of the second live moving image 41b while the first playback image 42a and the second live moving image 41b are displayed, stops display of the first playback image 42a and switches a display of the second live moving image 41b to a display of the second playback image 42b based on the second playback data. As a result, it is possible to perform reading out the first playback data to display the first playback image 42a in parallel with storing the second playback data on the second live moving image 41b. Further, since the playback data on the latest live moving image 41 is always accumulated, it is possible to start the display of the playback image 42 regarding the latest live moving image 41 at any time.

Further, HDD 113 as a non-transitory computer-readable storage medium according to the present embodiment stores a program 113a causing a hardware processor 100 as a computer provided in an ultrasound diagnostic apparatus 1 to perform generating sound ray data on ultrasound based on a reception signal from an ultrasound probe, the ultrasound being transmitted from the ultrasound probe to a subject and reflected in the subject, and controlling to display an ultrasound image based on the sound ray data on a display 19a. The program causes the hardware processor 100 to perform operations including: storing the sound ray data used for displaying the ultrasound images or processed sound ray data obtained by processing the sound ray data in a storage 16 as playback data which is used for controlling playback and display of the ultrasound images; and performing two-screen display control for displaying a live moving image 41 and a playback image 42 on the display 19a side by side, the live moving image 41 including ultrasound images being sequentially updated and displayed based on the sound ray data which is newly generated, and the playback image 42 being ultrasound images replayed and displayed based on the playback data on the live moving image 41. If an input receiver receives a first input operation for changing display control on the live moving image 41 in the two-screen display control, display control on the live moving image 41 is changed according to the first input operation, and if the input receiver receives a second input operation for changing display control on the playback image 42 in the two-screen display control, display control on the playback image 42 is changed according to the second input operation while continuing displaying the live moving image 41.

As a result, in the two-screen display where the live moving image 41 and the playback image 42 are displayed side by side, it is possible to operate the playback image by the second input operation during display of the live moving image 41 which is in an operable state by the first input operation. Therefore, the ultrasound diagnostic apparatus 1 can be used in a more flexible way.

### Second Embodiment

Next, the second embodiment of the present invention will be described. The ultrasound diagnostic apparatus 1 according to the present embodiment is different from that of the first embodiment in that the sound ray data (RAW data) generated in the receiver 13 is stored in the storage 16 before being processed by the signal processor 15 as the playback data. Hereinafter, differences from the first embodiment will be mainly described.

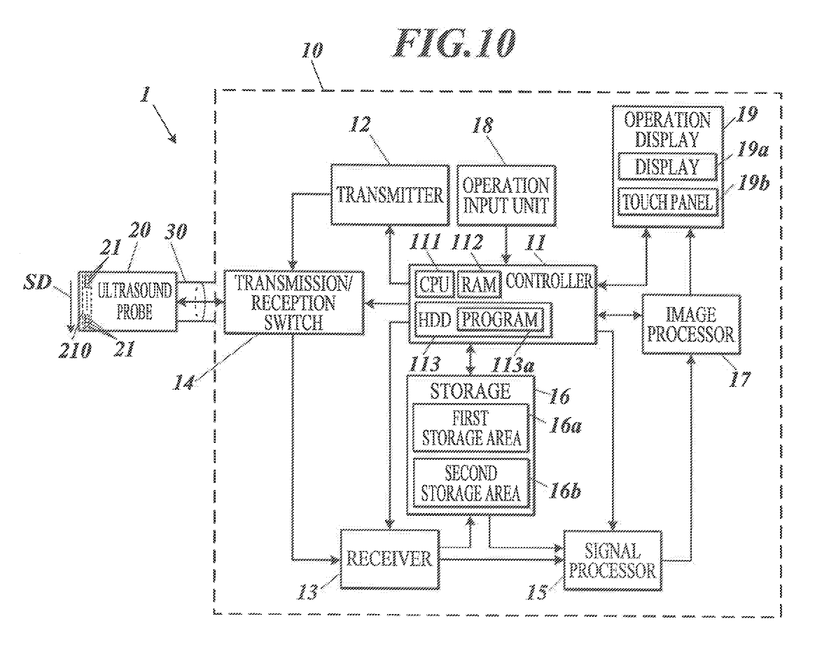

FIG. 10 is a block diagram showing a main functional configuration of the ultrasound diagnostic apparatus 1 according to the present embodiment.

In the ultrasound diagnostic apparatus 1 of the present embodiment, the sound ray data generated by the receiver 13 is also output to the storage 16 in addition to the signal processor 15. The sound ray data output to the storage 16 is stored in one of the first storage area 16a and the second storage area 16b depending on the state of the two-screen display and is accumulated as playback data.

When the playback image 42 is displayed on the basis of this playback data, the playback data (sound ray data, raw data) is read from the storage 16 and output to the signal processor 15. In the signal processor 15, various kinds of data processing is performed on the sound ray data for the purpose of adjusting the image quality of the ultrasound image to generate the processed sound ray data. The processed sound ray data is converted into image data in the image processor and used for display by the display 19a.

As described above, since the playback data of the present embodiment is sound ray data which has not been processed by the signal processor 15, it is possible to adjust the image quality (change of gain, dynamic range, etc.) of the playback image 42 by changing the parameter setting regarding the data processing by the signal processor 15.

For example, if the operation input receiver 18 or the touch panel 19b receives an input operation (second input operation) for changing the parameter setting in the data processing by the signal processor 15 while the moving image of the playback image 42 is displayed, the signal processor 15 performs data processing on the basis of the parameter setting changed by the input operation. As a result, the image quality of the moving image of the playback image 42 can be adjusted later. Likewise, image quality of the still image playback image 42 can also be adjusted at a desired timing

If there is no instruction by the operator on changing the parameter setting, in the data processing regarding the playback image 42, the signal processor 15 reads the parameter setting of the data processing used in the display processing of the live moving image 41 corresponding to the playback image 42 and performs data processing on the playback image 42 also on the basis of the same parameter setting.

As described above, the ultrasound diagnostic apparatus 1 according to the second embodiment includes a hardware processor 100 which generates the processed data by performing data processing on the sound ray data, the data processing regarding at least one of compression of data amount and adjustment of image quality of the ultrasound images, generates image data on the ultrasound images on a basis of the processing data, displays the ultrasound images on the display 19a on a basis of the image data, and stores the sound ray data which is raw data in the storage 16 as the playback data. By storing sound ray data (raw data) before being processed by the signal processor 15 as the processed data generating means as the playback data in this way, it is possible to change parameter settings regarding the data processing by the signal processor 15 when the playback image 42 is displayed, and to adjust the image quality (change of gain, dynamic range, etc.) of the playback image 42. As a result, it is possible to save time and labor for executing the ultrasound scan again, because an ultrasound image (playback image 42) of a required image quality may be obtained even if a live moving image 41a of a desired image quality is not obtained.

Further, if the input receiver receives the second input operation for changing a parameter setting regarding the data processing in generating the processed data on the playback image 42, the hardware processor 100 performs the data processing on a basis of a parameter setting changed by the second input operation. As a result, it is possible to easily adjust the image quality of the playback image 42 while displaying the live moving image 41 and the playback image 42.

Further, in generating the processed data on the playback image 42, the hardware processor 100 performs the data processing on a basis of a parameter setting which is equivalent to a parameter setting used in the data processing for generating the processed data on the live moving image 41. As a result, it is possible to easily display the playback image 42 with the same image quality as that of the live moving image 41.

The present invention is not limited to the above embodiments and modifications, and various changes may be made.

For example, the operation buttons displayed on the ultrasound diagnostic screen in FIG. 3 and the like and the physical operation unit in the operation input receiver 18 in FIG. 9 are not limited to those shown in the above embodiments and modifications. For example, when an operation button for storing the playback image 42 is displayed and the touch operation for selecting the operation button is performed, the image data of the ultrasound image displayed as the playback image 42 may be stored in the storage 16 or the HDD 113. Further, any one of the operation buttons of the operation input receiver 18 may be assigned for a function for storing the playback image 42.

Further, the number of the storage areas of the storage 16 is not limited to two (a first storage area 16a and the second storage area 16b), but may be three or more. According to such a configuration, every time the stop (freeze) processing of the live moving image 41 is performed, it is possible to sequentially switch the storage areas to store the playback data, so that the playback image regarding one of the multiple live moving images 41 of the past can be selected and displayed.

Further, in the above embodiments and modifications, the processed sound ray data (first embodiment) just before conversion processing to image data by the image processor 17, or the sound ray data immediately after being generated by the receiver (RAW data) (the second embodiment) is stored in the storage as the playback data, however, the present invention is not limited to this. Sound ray data at any stage or processed data (processed sound ray data and image data). The sound ray data or processed data (processed sound ray data or image data) at any step from being generated by the receiver to input to the display 19a may be used as the playback data.

Further, instead of the storage 16 inside the ultrasound diagnostic apparatus 1, a storage device provided outside the ultrasound diagnostic apparatus 1 may be used.

Further, in the embodiments and modifications described above, the present invention is applied to the ultrasound diagnostic apparatus 1 including the operation display 19 and the ultrasound probe 20. However, the present invention is not limited to this. The present invention may be applied to an ultrasound diagnostic apparatus constituted by an ultrasound diagnostic apparatus main body 10 where one or both of the operation display 19 and the ultrasound probe 20 are detachably attached.

In one embodiment, the input receiver has a physical operation unit including at least one of a rotary input key capable of receiving the input operation of rotation, a toggle switch capable of receiving the input operation of tilting to one side and another side along a predetermined direction, and a push button capable of receiving the input operation of pressing down. If the playback image is not displayed on the display, the physical operation unit receives an input operation regarding a predetermined function other than the second input operation.

In one embodiment, the hardware processor (i) generates the processed data by performing data processing on the sound ray data, the data processing regarding at least one of compression of data amount and adjustment of image quality of the ultrasound images, (ii) generates image data on the ultrasound images on a basis of the processing data, (iii) displays the ultrasound images on the display on a basis of the image data, and (iv) stores the processed data in the storage as the playback data.

In one embodiment, the hardware processor (i) generates the processed data by performing data processing on the sound ray data, the data processing regarding at least one of compression of data amount and adjustment of image quality of the ultrasound images, (ii) generates image data on the ultrasound images on a basis of the processing data, (iii) displays the ultrasound images on the display on a basis of the image data, and (iv) stores the sound ray data which is raw data in the storage as the playback data.

In one embodiment, if the input receiver receives the second input operation for changing a parameter setting regarding the data processing in generating the processed data on the playback image, the hardware processor performs the data processing on a basis of a parameter setting changed by the second input operation.

In one embodiment, in generating the processed data on the playback image, the hardware processor performs the data processing on a basis of a parameter setting which is equivalent to a parameter setting used in the data processing for generating the processed data on the live moving image.

In one embodiment, the hardware processor (i) stores a first playback data on a first live moving image in a first storage area in the storage, (ii) stores a second playback data on a second live moving image in a second storage area which is different from the first storage area in the storage, the second live moving image being displayed while the first playback image is displayed on a basis of the first playback data, and (iii) if the input receiver receives a predetermined input operation for stopping display of the second live moving image while the first playback image and the second live moving image are displayed, stops display of the first playback image and switches a display of the second live moving image to a display of the second playback image based on the second playback data.

Although embodiments of the present invention have been described and illustrated in detail, it is clearly understood that the same is by way of illustration and example only and not limitation, the scope of the present invention should be interpreted by terms of the appended claims

Japanese Patent Application No. 2018-006064 filed on Jan. 18, 2018, including description, claims, drawings, and abstract of the entire disclosure is incorporated herein by reference in its entirety.

1. An ultrasound diagnostic apparatus comprising:
a hardware processor which generates sound ray data on ultrasound based on a reception signal from an ultrasound probe, the ultrasound being transmitted from the ultrasound probe to a subject and reflected in the subject and which displays ultrasound images based on the sound ray data on a display; and
an input receiver which receives an input operation for changing display control on the ultrasound images by the hardware processor,
wherein the hardware processor stores the sound ray data used for displaying the ultrasound images or processed data obtained by processing the sound ray data in a storage, as playback data to be used for controlling playback and display of the ultrasound images,
wherein the hardware processor performs two-screen display control for displaying a live moving image and a playback image on the display side by side, the live moving image including ultrasound images being sequentially updated and displayed based on the sound ray data which is newly generated, and the playback image being ultrasound images replayed and displayed based on the playback data on the live moving image, and
wherein, in the two-screen display control,
if the input receiver receives a first input operation for changing display control on the live moving image, the hardware processor changes display control on the live moving image according to the first input operation, and
if the input receiver receives a second input operation for changing display control on the playback image, the hardware processor changes display control on the playback image according to the second input operation while continuing displaying the live moving image.

2. The ultrasound diagnostic apparatus according to claim 1,
wherein the hardware processor displays one of the ultrasound images of multiple frames based on the playback data as the playback image, and
wherein, if the input receiver receives the second input operation for changing frame of the playback image, the hardware processor changes frame of the playback image to be displayed according to the second input operation.

3. The ultrasound diagnostic apparatus according to claim 1,
wherein the hardware processor displays a moving image of the playback image by sequentially updating an image to be displayed among the ultrasound images of multiple frames based on the playback data.

4. The ultrasound diagnostic apparatus according to claim 3,
wherein, if the input receiver receives the second input operation for changing playback speed of a moving image of the playback image, the hardware processor changes playback speed of a moving image of the playback image according to the second input operation.

5. The ultrasound diagnostic apparatus according to claim 3,
wherein, if the input receiver receives the second input operation for specifying a playback range of a moving image of the playback image, the hardware processor displays an moving image of the playback image within the playback range according to the second input operation.

6. The ultrasound diagnostic apparatus according to claim 3,
wherein, if the input receiver receives a predetermined input operation under the two-screen display control, the hardware processor stores, in the storage, image data of moving images including a moving image of the live moving image and a moving image of the playback image.

7. The ultrasound diagnostic apparatus according to claim 1,
wherein the input receiver has a touch panel superimposed on a display screen of the display, and
wherein the second input operation is a touch operation on the touch panel.

8. The ultrasound diagnostic apparatus according to claim 2,
wherein the input receiver has a touch panel superimposed on a display screen of the display,
wherein the second input operation is a touch operation on the touch panel, and
wherein the hardware processor changes a frame of the playback image to be displayed according to a flick operation and a direction of the flick operation, the flick operation being performed on an area where the touch panel is superimposed on the playback image.

9. The ultrasound diagnostic apparatus according to claim 3,
wherein the input receiver has a touch panel superimposed on a display screen of the display,
wherein the second input operation is a touch operation on the touch panel, and
wherein the hardware processor starts or stops display of a moving image of the playback image according to a flick operation and a direction of the flick operation, the flick operation being performed on an area where the touch panel is superimposed on the playback image.

10. The ultrasound diagnostic apparatus according to claim 4,
wherein the input receiver has a touch panel superimposed on a display screen of the display,
wherein the second input operation is a touch operation on the touch panel, and
wherein the hardware processor changes playback speed of a moving image of the playback image according to a flick operation and a direction of the flick operation, the flick operation being performed on an area where the touch panel is superimposed on the playback image.

11. The ultrasound diagnostic apparatus according to claim 7,
wherein the hardware processor displays at least one operation target image on the display, the operation target image indicating a target on which the contact operation is to be performed, and
wherein the second input operation is a touch operation on a predetermined operation target image of the at least one operation target image.

12. The ultrasound diagnostic apparatus according to claim 11, wherein the hardware processor displays the predetermined operation target image in a vicinity of the playback image.

13. The ultrasound diagnostic apparatus according to claim 11, wherein the hardware processor displays the predetermined operation target image in a state superimposed on the playback image.

14. The ultrasound diagnostic apparatus according to claim 11, wherein, if a touch operation on the touch panel has not been performed for a predetermined time, the hardware processor deletes the predetermined operation target image.

15. The ultrasound diagnostic apparatus according to claim 11, wherein, if a touch operation is performed on an area superimposed on the playback image or on a vicinity of the playback image in the touch panel while the predetermined operation target image is not displayed, the hardware processor displays the predetermined operation target image.

16. The ultrasound diagnostic apparatus according to claim 11, wherein, if display of the playback image is deleted or if a display of the playback image is switched to a display of another image, the hardware processor deletes the predetermined operation target image.

17. The ultrasound diagnostic apparatus according to claim 2,
wherein the input receiver has a rotary input key capable of receiving the input operation of rotation, and
wherein, if the second input operation of rotating the rotary input key is received, the hardware processor changes a frame of the playback image to be displayed according to a rotation amount and a rotation direction of the rotary input key.

18. The ultrasound diagnostic apparatus according to claim 2,
wherein the input receiver has a toggle switch capable of receiving the input operation of tilting to one side and another side along a predetermined direction, and
wherein, if the second input operation of tilting the toggle switch is received, the hardware processor changes a frame of the playback image to be displayed according to a tilt of the toggle switch and a direction of the tilt.

19. The ultrasound diagnostic apparatus according to claim 3,
wherein the input receiver has a push button capable of receiving the input operation of pressing down, and
wherein, if the second input operation of pressing the push button is received, the hardware processor starts or stops display of a moving image of the playback image according to pressing of the push button.

20. (canceled)

21. (canceled)

22. (canceled)

23. (canceled)

24. (canceled)

25. (canceled)

26. A non-transitory computer-readable storage medium storing a program causing a computer provided in an ultrasound diagnostic apparatus to perform:
generating sound ray data on ultrasound based on a reception signal from an ultrasound probe, the ultrasound being transmitted from the ultrasound probe to a subject and reflected in the subject, and
controlling to display ultrasound images based on the sound ray data on a display,
wherein the program causes the computer to perform:
storing the sound ray data used for displaying the ultrasound images or processed data obtained by processing the sound ray data in a storage as playback data which is used for controlling playback and display of the ultrasound images; and
performing two-screen display control for displaying a live moving image and a playback image on the display side by side, the live moving image including ultrasound images being sequentially updated and displayed based on the sound ray data which is newly generated, and the playback image being ultrasound images replayed and displayed based on the playback data on the live moving image, and
wherein, if a predetermined input receiver receives a first input operation for changing display control on the live moving image in the two-screen display control, display control on the live moving image is changed according to the first input operation, and
wherein, if the input receiver receives a second input operation for changing display control on the playback image in the two-screen display control, display control on the playback image is changed according to the second input operation while continuing displaying the live moving image.
